(19) United States
(12) Patent Application Publication
(10) Pub. No.: US 2012/0197131 A1
(43) Pub. Date: Aug. 2, 2012

(54) PROBE-MOUNTED ULTRASOUND SYSTEM CONTROL INTERFACE

(75) Inventors: Emil Markov Georgiev, Hartland, WI (US); Menachem Halmann, Bayside, WI (US); Douglas Dietz, Pewaukee, WI (US); Yalan Yang, Wuxi (CN); Jeffery Scott Peiffer, Waukesha, WI (US); Robert Andrew Meurer, Waukesha, WI (US); Yu Ma, Wuxi (CN); Jonathan Alan Bowman, Montgomery, AL (US); Sarah Cooper-Davis, Milwaukee, WI (US); Renee Stasiewicz, Dousman, WI (US); Betty Edith Francoise Hohmann, Buc (FR); Adel Dotzler, Wauwatosa, WI (US); David Bong, Bruce, WI (US); William Arnold, Wilmette, IL (US); Alfredo Tirado, Orlando, FL (US); Tomer Begaz, Milwaukee, WI (US); Eric Shepard, Woodstock, MD (US); Steven Clendenen, Ponte Vedra Beach, FL (US)

(73) Assignee: GENERAL ELECTRIC COMPANY, Schenectady, NY (US)

(21) Appl. No.: 13/019,139

(22) Filed: Feb. 1, 2011

(51) Int. Cl. A61B 8/00

(52) U.S. Cl. 600/459

(57) ABSTRACT

In one embodiment, an ultrasound probe is provided including a probe body with a sensing face arranged to be held in contact with a subject by a user. The probe also provides a tactile interface located on a side of the probe body sufficiently close to the sensing face so that a user can depress the tactile interface with a finger while the probe is grasped by the user via the thumb of the same hand and at least two other fingers of the same hand rest in contact with the subject. The probe includes at least one switch which is activated by depression of the tactile interface.

### BACKGROUND OF THE INVENTION

The subject matter disclosed herein relates generally to ultrasound systems, and, more particularly, to a probe-mounted ultrasound system control interface.

Ultrasound systems may be used to examine and study anatomical structures, and to assist operators, typically radiologists and surgeons, in performing medical procedures. These systems typically include ultrasound scanning devices, such as ultrasound probes, that transmit pulses of ultrasound waves into the body. Acoustic echo signals are generated at interfaces in the body in response to these waves. These echo signals are received by the ultrasound probe and transformed into an electrical signal that is used to produce an image of the body part under examination. This image may be displayed on a display device.

When an ultrasound system is used to assist an operator in performing a medical procedure, the operator may hold an ultrasound probe in one hand, while holding a medical instrument in their other hand. This may make it difficult for the operator to adjust settings on the ultrasound system because both of the operator's hands are busy positioning devices. Unfortunately, an additional operator may be needed to assist in adjusting operating settings of the ultrasound system during such a procedure, or to hold the ultrasound probe, or to perform other tasks due to the inability to access controls while holding the probe and another medical instrument. Therefore, a system that enables an operator to perform a medical procedure using an ultrasound probe in one hand and a medical instrument in another hand without an additional operator may decrease the number of operators necessary for the procedure, improve controllability of the process, and render it more intuitive.

### BRIEF DESCRIPTION OF THE INVENTION

In one embodiment, an ultrasound probe includes a probe body having a sensing face configured to be held in contact with a subject by a user. A tactile interface is disposed on a side of the probe body sufficiently close to the sensing face to permit depression of the tactile interface by a finger of a hand of the user while the probe is grasped by the user via the thumb of the same hand, and at least two other fingers of the same hand rest in contact with the subject. At least one switch is activated by depression of the tactile interface.

In another embodiment, an ultrasound system includes a probe having a probe body and a sensing face configured to be held in contact with a subject by a user. A tactile interface is disposed on a side of the probe body sufficiently close to the sensing face to permit depression of the tactile interface by a finger of a hand of the user while the probe is grasped by the user via the thumb of the same hand, and at least two other fingers of the same hand rest in contact with the subject. At least one switch is activated by depression of the tactile interface. The ultrasound system also includes a system controller configured to receive signals from the at least one switch and to alter an ultrasound imaging function based upon the signals.

In a further embodiment, an ultrasound system includes a probe having a probe body and a sensing face configured to be held in contact with a subject by a user. A tactile interface is disposed on a side of the probe body sufficiently close to the sensing face to permit depression of the tactile interface by a finger of a hand of the user while the probe is grasped by the user via the thumb of the same hand, and at least two other fingers of the same hand rest in contact with the subject. At least one switch is activated by depression of the tactile interface. The ultrasound system also includes a system controller configured to receive signals from the at least one switch and to select one of a plurality of ultrasound imaging functions based upon the signals, and a user viewable interface coupled to the system controller and configured to display a plurality of settings corresponding to the plurality of ultrasound imaging functions selected by activation of the at least one switch.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 is a block diagram of an ultrasound system in accordance with aspects of the present disclosure;

FIG. 2 is a perspective view of an embodiment of an ultrasound probe with a tactile interface;

FIG. 3 is a perspective view of an embodiment of an ultrasound probe being held by a user;

FIG. 4 is a cross-section view of an embodiment of an ultrasound probe tactile interface;

FIG. 5 is a cross-section view of the embodiment of the ultrasound probe tactile interface of FIG. 4 with the top button depressed;

FIG. 6 is a cross-section view of the embodiment of the ultrasound probe tactile interface of FIG. 4 with the middle button depressed;

FIG. 7 is a cross-section view of the embodiment of the ultrasound probe tactile interface of FIG. 4 with the bottom button depressed;

FIG. 8 is a view of an embodiment of an ultrasound system configured to display a menu;

FIG. 9 is a view of the menu of FIG. 8 with a change made to the selected menu option;

FIG. 10 is a view of an embodiment of the ultrasound system of FIG. 9 configured to display a level indicator; and

FIG. 11 is a view of the level indicator of FIG. 10 with the setting level of the level indicator changed.

### DETAILED DESCRIPTION OF THE INVENTION

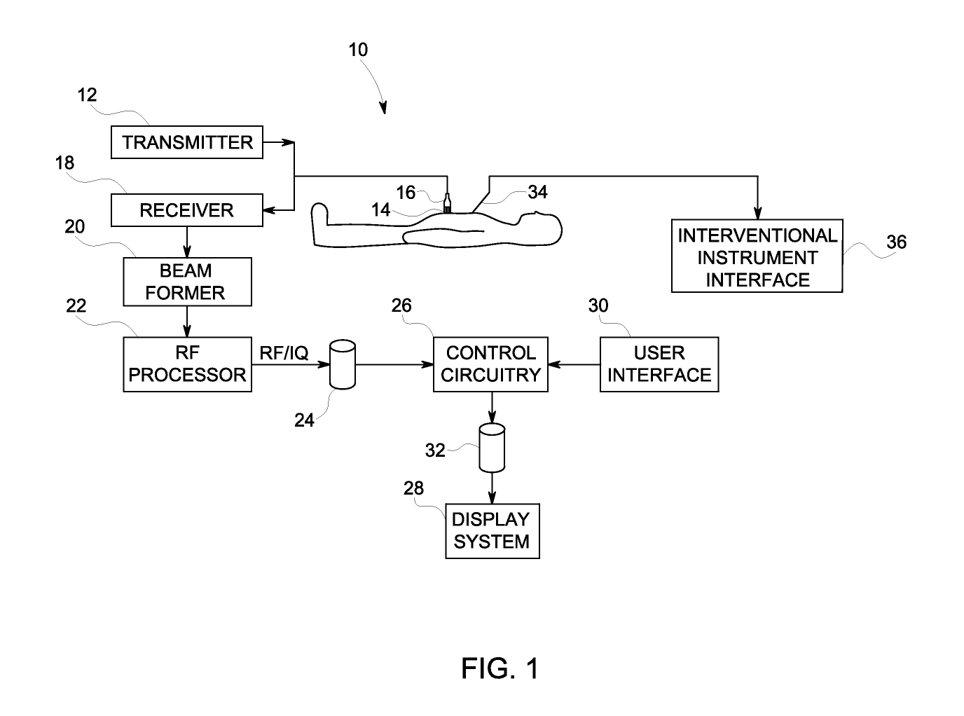

FIG. 1 is a block diagram of an ultrasound system 10 that may be used, for example, to acquire and process ultrasonic images. The ultrasound system 10 includes a transmitter 12 that drives one or more arrays of elements 14 (e.g., piezoelectric crystals) within or formed as part of a probe 16 to emit pulsed ultrasonic signals into a body or volume. A variety of geometries may be used and one or more transducers may be provided as part of the probe 16. The pulsed ultrasonic signals are back-scattered from density interfaces and/or structures, for example, in a body, like blood cells or muscular tissue, to produce echoes that return to the elements 14. The echoes are received by a receiver 18 and provided to a beam former 20. The beam former 20 performs beamforming on the received echoes and outputs an RF signal. The RF signal is then processed by an RF processor 22. The RF processor 22 may include a complex demodulator (not shown) that demodulates the RF signal to form IQ data pairs representative of the echo signals. The RF or IQ signal data then may be routed directly to an RF/IQ buffer 24 for storage (e.g., temporary storage).

The ultrasound system 10 also includes control circuitry 26 to process the acquired ultrasound information (i.e., RF signal data or IQ data pairs) and to prepare frames of ultrasound information for display on a display system 28. The control circuitry 26 may be adapted to perform one or more processing operations according to a plurality of selectable ultrasound modalities on the acquired ultrasound information. Acquired ultrasound information may be processed in real-time during a scanning session as the echo signals are received. Additionally or alternatively, the ultrasound information may be stored temporarily in the RF/IQ buffer 24 during a scanning session and processed in less than real-time in a live or off-line operation.

A user interface 30 may be used to control operation of the ultrasound system 10. The user interface 30 may be any suitable device for receiving user inputs to control, for example, the type of scan or type of transducer to be used in a scan. As such, the user interface may include a keyboard, mouse, and/or touch screen.

The ultrasound system 10 may continuously acquire ultrasound information at a desired frame rate, such as rates exceeding fifty frames per second, which is the approximate perception rate of the human eye. The acquired ultrasound information may be displayed on the display system 28 at a slower frame-rate. An image buffer 32 may be included for storing processed frames of acquired ultrasound information that are not scheduled to be displayed immediately. In one embodiment, the image buffer 32 is of sufficient capacity to store at least several seconds of frames of ultrasound information. The frames of ultrasound information may be stored in a manner to facilitate retrieval thereof according to their order or time of acquisition. The image buffer 32 may comprise any known data storage medium.

An interventional instrument 34 may be used as part of the ultrasound system 10 to enable a user to perform a medical procedure on a patient while collecting ultrasound information from the probe 16. The interventional instrument 34 may include sensors, gyroscopes, and/or accelerometers to aid in determining position information of the interventional instrument 34. An interventional instrument interface 36 receives electrical signals from the interventional instrument 34 and converts these signals into information such as position data, orientation data, trajectory data, or other sensor information. Furthermore, the probe 16 may include a tactile interface located to enable the user to adjust ultrasound settings with the hand holding the probe 16 and thus enable the user to use an interventional instrument with their other hand.

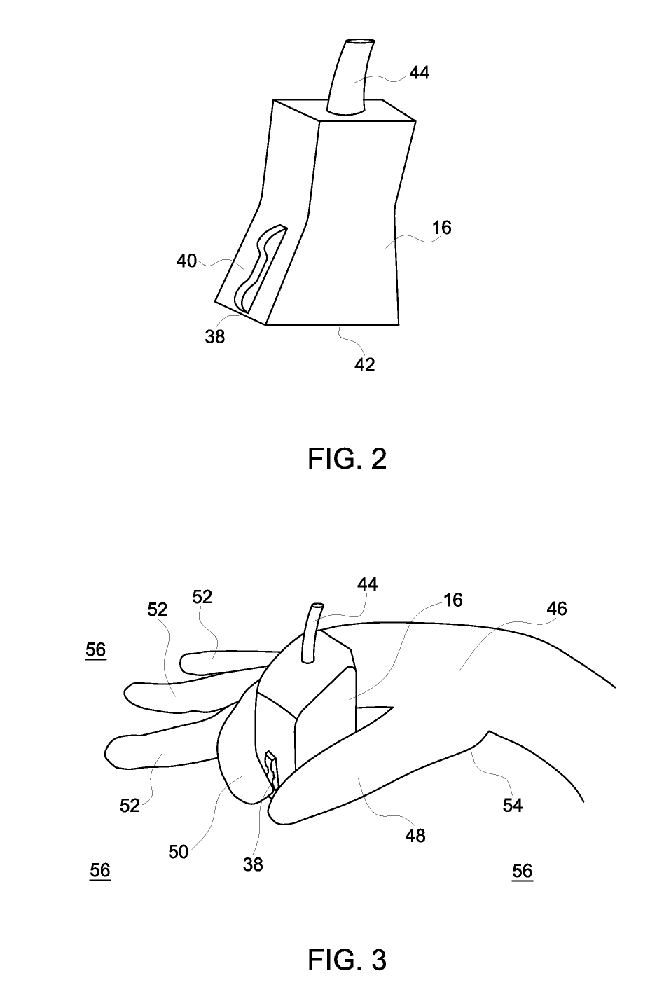

FIG. 2 is a perspective view of an embodiment of an ultrasound probe 16 with a tactile interface 38. The ultrasound probe 16 is designed to be held by an operator and placed in contact with a subject to acquire ultrasound images. The tactile interface 38 is located on a side 40 of the probe 16 close to a sensing face 42 of the probe 16. The tactile interface 38 may be touch activated, such as by a hand or finger of the operator of the ultrasound probe 16. The ultrasound probe 16 and the tactile interface 38 may be generally bilaterally symmetrical to permit the operator to grasp the probe 16 and operate the tactile interface 38 using either their left or right hand. The sensing face 42 of the probe 16 receives ultrasonic echoes used to produce ultrasound images. Signals relating to the ultrasonic echoes are communicated to the ultrasound system via a cable 44.

As illustrated, the tactile interface 38 may include a button with a rocker that may be depressed to activate one or more switches. Alternatively, other contemplated embodiments may have a tactile interface 38 with multiple buttons, a scroll wheel, a finger-operated joystick, or other suitable interfaces that may be located on the ultrasound probe 16. Moreover, when buttons, regions or zones of activation are used, these may comprise any suitable technology, such as electromechanical (e.g., rocker) switches, membrane switches, inductive or capacitive contact switches, and so forth. Furthermore, the tactile interface 38 may have a generally rectangular footprint as shown with a button or buttons protruding from the face of the ultrasound probe 16, or the tactile interface 38 may use an alternate footprint and/or arrangement. For example, the tactile interface 38 may have a generally round, oval, square or triangular footprint. In addition, the tactile interface 38 may include a protecting seal to protect internal components of the probe 16 from contamination. Such seals may be provided over the probe and/or the interface, or may be built into the interface (e.g., beneath a cover or membrane).

FIG. 3 is a perspective view of an embodiment of an ultrasound probe 16 being held by a user. Although a right hand is depicted, a left hand may alternatively be used to hold the probe 16. A hand 46 of the user may hold the probe 16 using a thumb 48 on one side, and a forefinger 50 on the other side. The tactile interface 38 is positioned to enable the forefinger 50 to depress the button or buttons of the tactile interface 38. In addition, the tactile interface 38 is located sufficiently close to the face of the probe 16 to allow some or all of fingers 52 and a palm 54 of the user to rest on a subject 56. For example, the tactile interface 38 may be located approximately 2, 4, 6, 10, 12, 15, or 20 mm from the face of the probe 16. Thus, the user may be enabled to operate the tactile interface 38 using the forefinger 50 as well as hold the probe 16 at a desired location on the subject 56. Such a configuration may further enable the user to perform an interventional operation using an interventional instrument with the hand not holding the ultrasound probe 16. Moreover, it is preferred that the interface be accessible for use by the index finger and/or the middle finger, allowing the thumb, the palm of the thumb, and the other two fingers to remain in contact with the skin of the patient during use and activation of the interface.

FIG. 4 is a cross-section view of an embodiment of an ultrasound probe tactile interface 38. The tactile interface 38 includes a top button or region 58, a middle button or region 60, and a bottom button or region 62. Although reference is made in this discussion to “buttons” separately, these may be simply defined by regions or contours of a single element, such as a molded plastic cap. The buttons 58, 60, and 62 are connected to switches such that when the top button 58 is depressed, a top switch 64 is activated. Likewise when the middle button 60 is depressed, a middle switch 66 is activated, and when the bottom button 62 is depressed, a bottom switch 68 is activated. Springs 70 may be placed between the buttons 58, 60, and 62 and the switches 64, 66, and 68 to enable the buttons to return to a normal position when no longer depressed. The normal position (non-depressed) of the tactile interface 38 is illustrated by FIG. 4.

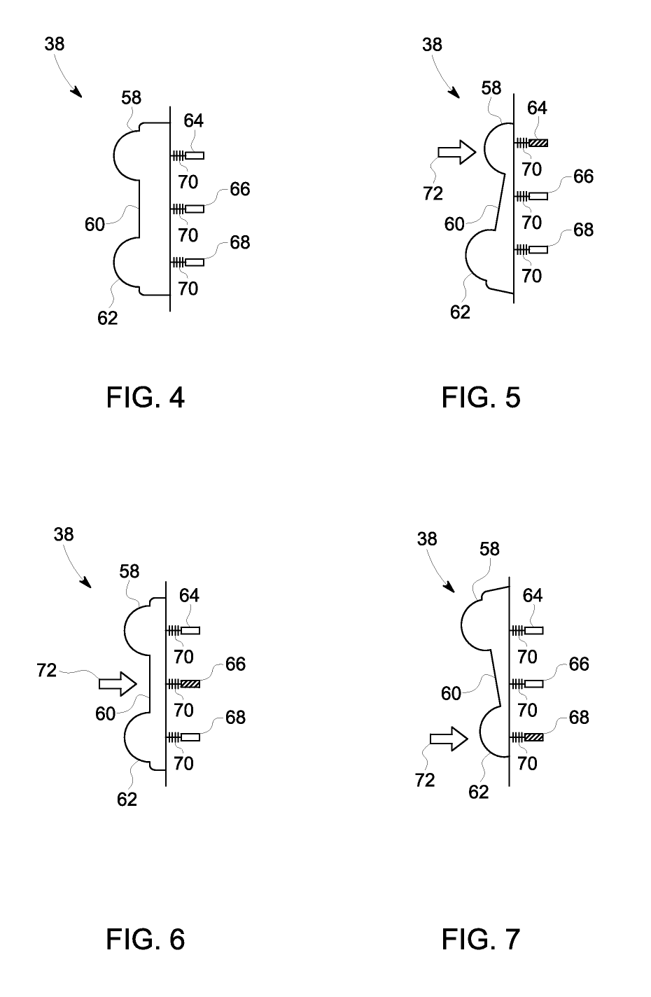

The buttons 58, 60, and 62 may be integrated with a rocker such that when the top button 58 is depressed, the rocker rocks to a position as depicted in FIG. 5, with the top button 58 depressed toward the top switch 64, and the bottom button 62 raised away from the bottom switch 68. Conversely, the rocker may rock to a position as depicted in FIG. 7, with the bottom button 62 depressed toward the bottom switch 68, and the top button 58 raised away from the top switch 64.

FIG. 5 is a cross-section view of the embodiment of the ultrasound probe's tactile interface 38 of FIG. 4 with the top button 58 depressed. An applied force 72, such as a finger applying pressure, is depicted causing the top button 58 to be depressed and the top switch 64 to be activated. When the applied force 72 is removed, the spring 70 may cause the tactile interface 38 to return to the normal position as illustrated in FIG. 4.

FIG. 6 is a cross-section view of the embodiment of the ultrasound probe tactile interface 38 of FIG. 4 with the middle button 60 depressed. Consequently, the middle switch 66 is activated when the applied force 72 causes the middle button 60 to be depressed. Again, when the applied force 72 is removed, the spring 70 may cause the tactile interface 38 to return to the normal position.

FIG. 7 is a cross-section view of the embodiment of the ultrasound probe tactile interface 38 of FIG. 4 with the bottom button 62 depressed. Here, the applied force 72 is depicted causing the bottom button 62 to be depressed and the bottom switch 68 to be activated. As in FIGS. 5 and 6, when the applied force 72 is removed, the spring 70 may cause the tactile interface 38 to return to the normal position depicted in FIG. 4.

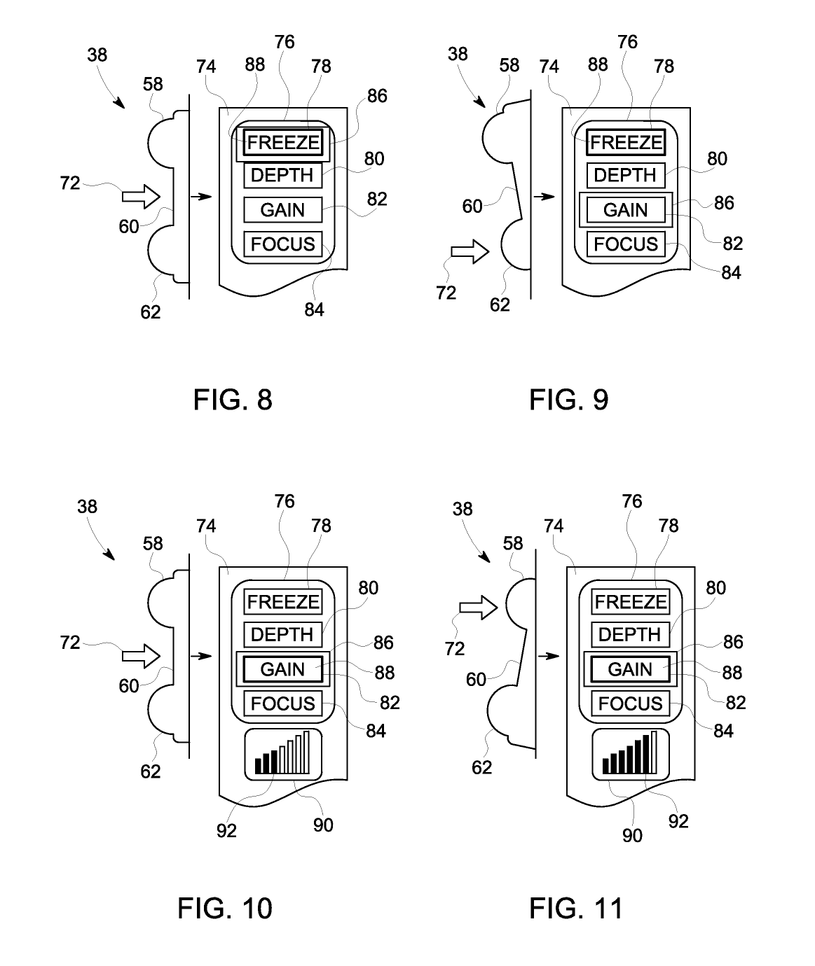

FIG. 8 is a view of an embodiment of an ultrasound system configured to display a menu when a user depresses a probe middle button. A tactile interface 38 is depicted with an applied force 72 depressing the middle button 60. When the button 60 is depressed and a switch is activated, signals may be sent from the ultrasound probe to control circuitry. The control circuitry may be coupled to a user viewable interface, such as a display with a display portion 74. The control circuitry may cause a menu 76 to be displayed on the portion of the display 74. Likewise, the display portion 74 may show the menu 76 concurrently with an ultrasound image. The menu 76 may be placed on the display so that the menu 76 does not cover the ultrasound image. Furthermore, the menu 76 may include any number of options which may be selected and/or altered by a user. As illustrated, the menu 76 may include: a first option 78, such as an option to freeze the ultrasound image being displayed; a second option 80, such as an option to change the depth settings of the ultrasound system; a third option 82, such as an option to change the gain settings of the ultrasound probe; and a fourth option 84, such as an option to change the focus of the ultrasound image being displayed. Other contemplated embodiments may include fewer or more than four options and may include such options as beam steering, print, and/or save functions.

A selection box 86 is depicted encompassing the first option 78. The selection box 86 may be used much like a cursor and may be moved to encompass one of the options 78, 80, 82, and 84 by depressing the top button 58 or the bottom button 62 until the desired option is encompassed. The selection box 86 enables the user to determine which option will be selected if the middle button 60 is depressed. The visual appearance of a selected option 88 may be altered to show that the option is selected. For example, FIG. 8 depicts the first option 78 as the selected option 88 by altering the like width of the first option 78 in relation to the other options. As will be appreciated, the number, operation, and order of options described may be different in other embodiments. For example, certain embodiment may have 2, 4, 6, or 8 options. Another embodiment may alter the number of buttons used to select and alter the options. For example, the tactile interface 38 may have two buttons with one button correlating to moving up among the options on the menu and one button correlating to moving down the options on the menu, while pressing a button twice quickly may select the option encompassed by the selection box 86.

FIG. 9 is a view of the menu 76 of FIG. 8 with a change in the selected menu option. The tactile interface 38 is displayed with the applied force 72 causing the bottom button 62 to be depressed. Pressing the bottom button 62 may cause the selection box 86 to move down from its current location to encompass the next option on the menu 76. For example, if the bottom button 62 is depressed two times, the selection box 86 may move from encompassing the first option 78 to encompassing the third option 82, as can be seen by comparing FIGS. 8 and 9. Conversely, if the top button 58 was depressed two times with the selection box positioned as in FIG. 9, the selection box 86 may move from encompassing the third option 82 to encompassing the first option 78.

FIG. 10 is a view of an embodiment of the ultrasound system of FIG. 9 configured to display a level indicator. The tactile interface 38 is illustrated with the applied force 72 depressing the middle button 60. With the menu 76 already on the display portion 74, depressing the middle button 60 may select the option encompassed by the selection box 86 by marking it as the selected option 88. If the selected option 88 is an option with adjustable settings, a level indicator 90 is displayed combined with the menu 76 on the display portion 74. The level indicator 90 indicates a setting level 92 of the current setting of the selected option 88. For example, the illustrated level indicator 90 depicts seven bars, with three bars darkened to show the current setting level 92 of three. The level indicator 90 enables the user to have a visual indication of the selected option's setting.

FIG. 11 is a view of the level indicator of FIG. 10 with the setting level of the level indicator changed. The tactile interface 38 is displayed with the applied force 72 depressing the top button 58. Depressing the top button 58 or the bottom button 62 while the level indicator 90 is displayed may result in the current setting level 92 increasing or decreasing respectively. For example, the illustrated level indicator 90 depicts the current setting level 92 of six out of seven bars. The current setting level 92 may have changed from the three bars in FIG. 10 to six bars in FIG. 11 by the user depressing the top button 58 three times. Conversely, the user may decrease the setting from six to three by depressing the bottom button 62 three times.

It should be understood that the particular arrangement of the interface, the menus navigated, and the sequence and logic of operation may, of course differ from those described, and these should be understood as examples only. Similarly, although three switches with corresponding interface manipulations are presently contemplated, an actual embodiment may include as few as a single button/switch, or more than three. It is also contemplated that particular menus, options, sequences of activation, and so forth may be adapted on particular system designs, depending upon the types of operations anticipated, the viewing or operating parameters available, and so forth.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. An ultrasound probe comprising:
a probe body having a sensing face configured to be held in contact with a subject by a user;
a tactile interface disposed on a side of the probe body sufficiently close to the sensing face to permit depression of the tactile interface by a finger of a hand of the user while the probe is grasped by the user via the thumb of the same hand and at least two other fingers of the same hand rest in contact with the subject; and
at least one switch activated by depression of the tactile interface.

2. The probe of claim 1, wherein the probe body and tactile interface are bilaterally symmetrical to permit grasping of the probe body and depression of the tactile interface by the left or right hand of the user.

3. The probe of claim 1, wherein the tactile interface comprises a button located to be depressed by a forefinger of the hand of the user.

4. The probe of claim 3, wherein the button comprises a rocker having at least one rocked position for activation of a corresponding switch.

5. The probe of claim 4, wherein the button comprises two rocked positions for activation of two corresponding switches.

6. The probe of claim 5, wherein the button comprises a depressed position intermediate the rocked positions for activation of a corresponding third switch.

7. An ultrasound system comprising:
a probe having a probe body and a sensing face configured to be held in contact with a subject by a user, a tactile interface disposed on a side of the probe body sufficiently close to the sensing face to permit depression of the tactile interface by a finger of a hand of the user while the probe is grasped by the user via the thumb of the same hand and at least two other fingers of the same hand rest in contact with the subject, and at least one switch activated by depression of the tactile interface; and
a system controller configured to receive signals from the at least one switch and to alter an ultrasound imaging function based upon the signals.

8. The system of claim 7, comprising a user viewable interface coupled to the system controller and configured to display at least one setting corresponding to the ultrasound imaging function altered by activation of the at least one switch.

9. The system of claim 8, wherein the tactile interface comprises positions configured to permit selection of an ultrasound imaging function to be altered, and at least two settings of each selectable ultrasound imaging function.

10. The system of claim 9, wherein a level indicator is displayed on the user viewable interface to show a setting level corresponding to the at least one setting of the ultrasound imaging function.

11. The system of claim 10, wherein the setting level of the level indicator is altered by activation of the at least one switch.

12. The system of claim 7, wherein the ultrasound imaging function comprises at least one of a gain setting, a focus setting, a depth setting, a freeze frame setting, and a beam steering function.

13. The system of claim 7, wherein the tactile interface is disposed to permit a palm of the same hand to rest in contact with the subject.

14. The system of claim 7, wherein the at least one switch comprises a protecting seal.

15. An ultrasound system comprising:
a probe having a probe body and a sensing face configured to be held in contact with a subject by a user, a tactile interface disposed on a side of the probe body sufficiently close to the sensing face to permit depression of the tactile interface by a finger of a hand of the user while the probe is grasped by the user via the thumb of the same hand and at least two other fingers of the same hand rest in contact with the subject, and at least one switch activated by depression of the tactile interface;
a system controller configured to receive signals from the at least one switch and to select one of a plurality of ultrasound imaging functions based upon the signals; and
a user viewable interface coupled to the system controller and configured to display a plurality of settings corresponding to the plurality of ultrasound imaging functions selected by activation of the at least one switch.

16. The system of claim 15, wherein the user viewable interface is configured to display a plurality of level indicators corresponding to the plurality of settings.

17. The system of claim 16, wherein a setting level of each of the plurality of level indicators is altered by activation of the at least one switch.

18. The system of claim 15, wherein the plurality of ultrasound imaging functions comprises at least one of a gain setting, a focus setting, a depth setting, a freeze frame setting, and a beam steering function.

19. The probe of claim 15, wherein the tactile interface comprises a button located to be depressed by a forefinger of the hand of the user.

20. The probe of claim 19, wherein the button comprises two rocked positions for activation of two corresponding switches.
